(19) United States
(12) Patent Application Publication
(10) Pub. No.: US 2015/0202098 A1
(43) Pub. Date: Jul. 23, 2015

(54) SANITARY NAPKIN STRUCTURE

(71) Applicant: TSENG, KUO-AN, Hsinchu City (TW)

(72) Inventor: TSENG, KUO-AN, Hsinchu City (TW)

(21) Appl. No.: 14/159,598

(22) Filed: Jan. 21, 2014

(51) Int. Cl. A61F 13/84

(52) U.S. Cl. CPC A61F 13/8405; A61F 2013/8438

(57) ABSTRACT

A sanitary napkin structure contains: a body, and the body include a surface layer formed on a top surface thereof, an additive layer defined below the surface layer, an absorbent isolation layer arranged below the additive layer, an absorbing layer defined below the absorbent isolation layer, and a bottom layer formed on a bottom surface of the body. The surface layer is woven from nano powder spinning fibers. The absorbent isolation layer is applied to absorb liquids and to prevent the liquids from leaking back to the surface layer, and the absorbing layer is used to absorb the liquids. The additive layer includes additives comprised of proteolytic enzyme. The bottom layer is breathable and liquid impervious. Thereby, the sanitary napkin structure is capable of avoiding bacterium infecting user.

### FIELD OF THE INVENTION

The present invention relates to a sanitary napkin structure which is capable of avoiding bacterium infecting user.

### BACKGROUND OF THE INVENTION

A conventional sanitary napkin is applied to absorb secretions or blood during menstruation period and contains a surface layer for contacting with user's skin. The surface layer is made of pulp or non-woven material.

However, the conventional sanitary napkin is airtight to grow bacterium and cause odor easily.

The present invention has arisen to mitigate and/or obviate the afore-described disadvantages.

### SUMMARY OF THE INVENTION

The primary object of the present invention is to provide a sanitary napkin structure which is capable of avoiding bacterium infecting user.

Another object of the present invention is to provide a sanitary napkin structure which inhibits bacterium, removes odor, and maintains aroma and dry.

To obtain the above objectives, a sanitary napkin structure provided by the present invention contains: a body, and the body includes a surface layer formed on a top surface thereof, an additive layer defined below the surface layer, an absorbent isolation layer arranged below the additive layer, an absorbing layer defined below the absorbent isolation layer, and a bottom layer formed on a bottom surface of the body.

The surface layer is woven from nano powder spinning fibers.

The absorbent isolation layer is applied to absorb liquids and to prevent the liquids from leaking back to the surface layer, and the absorbing layer is used to absorb the liquids.

The additive layer includes additives comprised of proteolytic enzyme.

The bottom layer is breathable and liquid impervious.

### BRIEF DESCRIPTION OF THE DRAWINGS

FIG. 1 is a perspective view showing the assembly of a sanitary napkin structure according to a preferred embodiment of the present invention.

FIG. 2 is a cross sectional view showing the assembly of a part of the sanitary napkin structure according to the preferred embodiment of the present invention.

FIG. 3 is a perspective view showing the assembly of a surface layer of the sanitary napkin structure according to the preferred embodiment of the present invention.

### DETAILED DESCRIPTION OF THE PREFERRED EMBODIMENTS

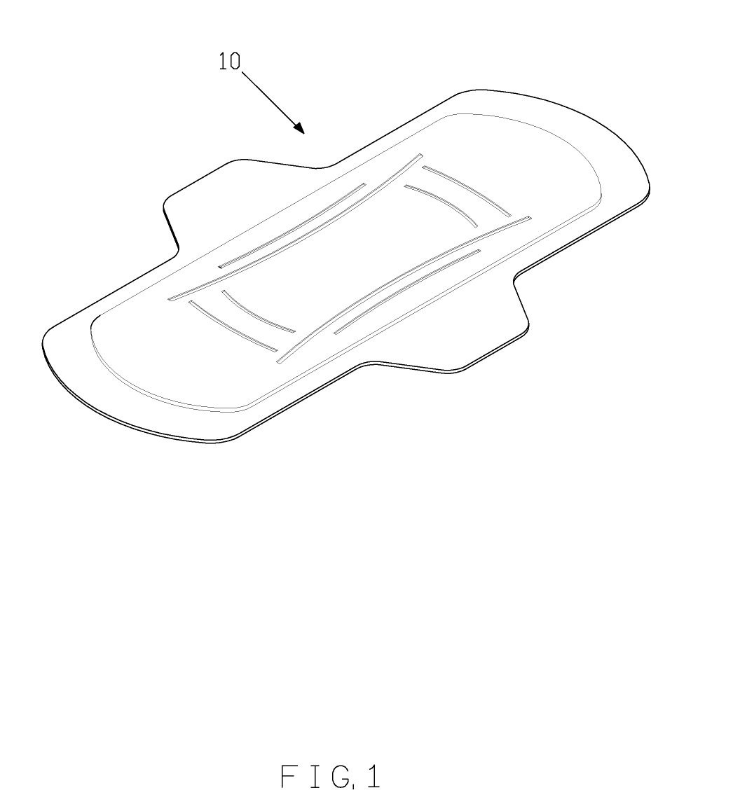

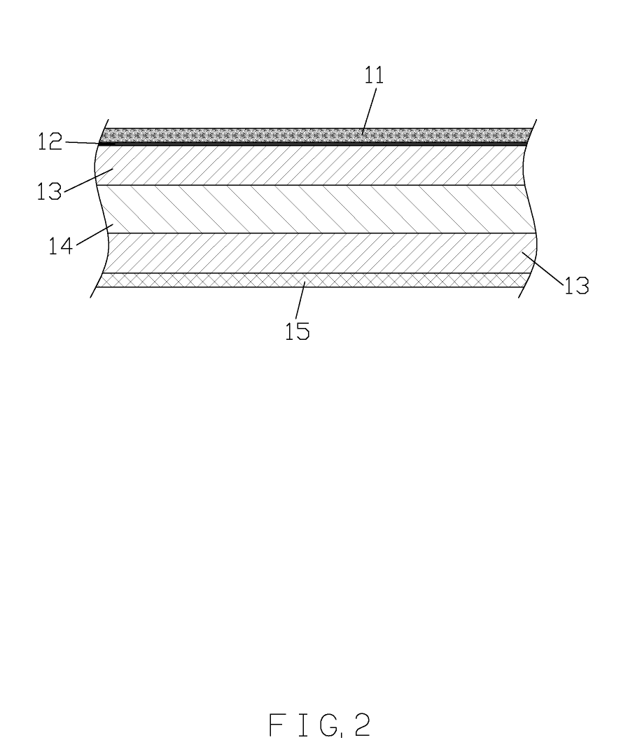

Referring to FIGS. 1 and 2, a sanitary napkin structure according to a preferred embodiment of the present invention comprises: a body 10, and the body 10 includes a surface layer 11 formed on a top surface thereof so as to contact with user's skin, an additive layer 12 defined below the surface layer 11, an absorbent isolation layer 13 arranged below the additive layer 12, an absorbing layer 14 defined below the absorbent isolation layer 13, and a bottom layer 15 formed on a bottom surface of the body 10. The bottom layer 15 is breathable and liquid impervious, the absorbent isolation layer 13 is applied to absorb liquids and to prevent the liquids from leaking back to the surface layer 11, and the absorbing layer 14 is used to absorb the liquids.

The additive layer 12 is defined between the surface layer 11 and the absorbent isolation layer 13 and includes additives comprised of proteolytic enzyme so as to inhibit bacterium, remove odor, and maintain aroma and dry. Thereby, the sanitary napkin structure is capable of avoiding bacterium infecting user.

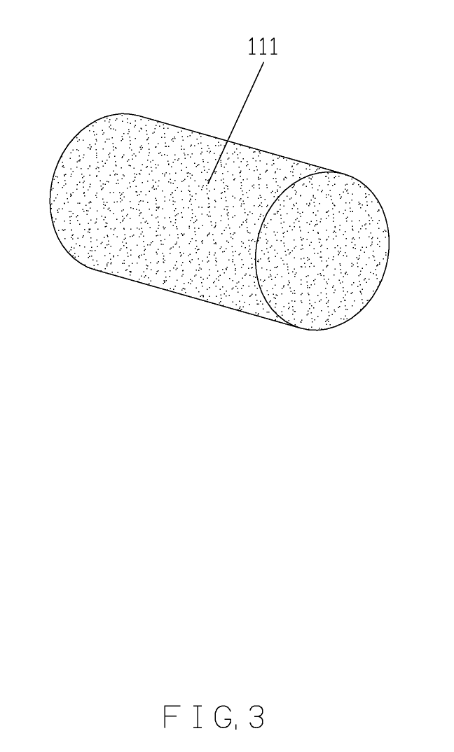

Referring further to FIG. 3, the surface layer 11 is woven from nano powder spinning fibers 111 so as to maintain clean and dry of the sanitary napkin structure.

While the preferred embodiments of the invention have been set forth for the purpose of disclosure, modifications of the disclosed embodiments of the invention and other embodiments thereof may occur to those skilled in the art. Accordingly, the appended claims are intended to cover all embodiments which do not depart from the spirit and scope of the invention.

What is claimed is:

1. A sanitary napkin structure comprising: a body, and the body including a surface layer formed on a top surface thereof, an additive layer defined below the surface layer, an absorbent isolation layer arranged below the additive layer, an absorbing layer defined below the absorbent isolation layer, and a bottom layer formed on a bottom surface of the body;
wherein the surface layer is woven from nano powder spinning fibers;
wherein the absorbent isolation layer is applied to absorb liquids and to prevent the liquids from leaking back to the surface layer, and the absorbing layer is used to absorb the liquids.

2. The sanitary napkin structure as claimed in claim 1, wherein the additive layer includes additives comprised of proteolytic enzyme.

3. The sanitary napkin structure as claimed in claim 1, wherein the bottom layer is breathable and liquid impervious.
